(19) United States
(12) Patent Application Publication
(10) Pub. No.: US 2017/0336392 A1
(43) Pub. Date: Nov. 23, 2017

(54) IMAGING METHODS TO ASSESS THE EFFICACY OF ANTICANCER DRUGS IN VITRO CANCER USING SPONTANEOUSLY- FORMING SPHEROIDS

(71) Applicants: Emmanuel A. Theodorakis, San Diego, CA (US); Mary L. Alpaugh, Morton, PA (US)

(72) Inventors: Emmanuel A. Theodorakis, San Diego, CA (US); Mary L. Alpaugh, Morton, PA (US)

(21) Appl. No.: 15/584,593

(22) Filed: May 2, 2017

Related U.S. Application Data
(60) Provisional application No. 62/330,513, filed on May 2, 2016.

(51) Int. Cl. G01N 33/50

(52) U.S. Cl. CPC G01N 33/5011

(57) ABSTRACT

The present invention relates to imaging methods to assess the efficacy of anticancer drugs in vitro using spontaneously-forming spheroids.

### CROSS-REFERENCE TO RELATED APPLICATIONS

The present application claims priority to U.S. Provisional Patent Application Ser. No. 62/330,513, filed May 2, 2016, hereby incorporated by reference in its entirety.

### STATEMENT REGARDING FEDERAL FUNDING

This invention was funded in part by NIH Grant Number CA 133002 and National Science Foundation instrumentation grants CHE9709183 and CHE0741968. The U.S. Government has certain rights with respect to this invention.

### FIELD OF THE INVENTION

The invention relates to a high-throughput-compatible method for using multicellular spheroids as an indicator of response to drug candidates.

### BACKGROUND OF THE INVENTION

It has been recognized that multi-cellular 3-D systems may represent a more relevant model of living tissue and can also provide valuable insights into processes that govern cancer progression, metastasis and drug resistance. Such methods include hanging drop, liquid overlays and microfabrication (e.g. cellular gel encapsulation). In general, these methods generate artificial environments that drive cells that under normal conditions would grow as a traditional 2-D culture, to form 3-D spheroid-like structures.

Methods that establish 3-D cell models include but are not limited to hanging drop, liquid overlays and microfabrication (e.g. cellular gel encapsulation). In general, these methods generate artificial environments that drive cells that under normal conditions would grow as a traditional 2-D monolayer culture, to form 3-D spheroid-like structures. Several factors, such as culture longevity that may fail to attain tissue-like phenotype, reproducibility of culture conditions and spheroid size, limit their translational utility as clinically relevant models. Additionally, these methods often generate a limited number of spheroids (e.g. hanging drop generates 1 spheroid/well) at a substantial cost, making these systems impractical for high-throughput drug screening and development.

Measurement of response in 3-D cultures is typically more involved in contrast to viability assays performed in 2-D monolayer cultures and remains a major impediment in utilization of 3-D models in high throughput screens. To assess response using 3-D models, fluorescent cell viability assays are analyzed using confocal microscopy with evaluation of live/dead cells of multiple focal planes and is paired with phase contrast analysis data of spheroid integrity, diameter and volume. These involved, time-consuming and costly methods are necessary to accurately assess treatment response in a multicellular spheroid. Accordingly, there is a need for rapid, high throughput screening of anti-cancer therapeutic compositions utilizing spheroids.

### SUMMARY OF THE INVENTION

The present disclosure relates to using spontaneously-forming multicellular spheroids as a platform in anticancer drug screening. Importantly, this invention provides a novel, rapid and reliable method to measure compound cytotoxicity on spheroids. Accordingly, in some embodiments, the present invention is directed to a method of screening cytotoxicity of a therapeutic compound. In some embodiments, the method comprises exposing a composition comprising a spontaneously-forming multicellular spheroid with the therapeutic compound for a period of time. In some embodiments, the spheroid is derived from a tumor. In some embodiments, the spheroid is derived from a tumor xenograft. In some embodiments, the spheroid is derived from a tumor biopsy. In some embodiments, the tumor is breast tumor, a colon tumor, a melanoma tumor, a lung tumor, a skin tumor, a pancreatic tumor, a liver tumor, a brain tumor, an ovarian tumor, a testicular tumor, a prostate tumor, a stomach tumor, a kidney tumor, a tracheal tumor, an oral tumor, or an esophageal tumor. In some embodiments, the tumor is an inflammatory breast cancer tumor. In some embodiments, the spheroid over-expresses at least one of E-cadherein, alpha-catenin, beta-catenin or combinations thereof; or another innate molecular determinant that induces spheroidal morphology. In some embodiments, the spheroid comprises an organoid. In some embodiments, the spheroid comprises a mammosphere. In some embodiments, the spheroid is a spheroid^{MARY-X}. In some embodiments, the spheroid is seeded in a multi-well plate. In some embodiments, the spheroid is seeded with at least one additional spheroid in a multi-well plate, e.g. at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 250, at least 300, at least 350, at least 400, at least 450, at least 500, or seeded with greater than at least 500 additional spheroids in a multi-well plate.

In some embodiments, the therapeutic compound comprises one of a peptide, polypeptide, nucleic acid, or a small molecule. In some embodiments, the therapeutic compound is a chemotherapeutic compound. In some embodiments, the therapeutic compound comprises a synthetic analog of a known compound. In other embodiments, the therapeutic compound comprises a natural product or analog of a known compound. In some embodiments, the therapeutic compound is an analog of gambogic acid. In some embodiments, the therapeutic compound comprises one of CR142, CR135, MAD44, and MAD28. In some embodiments, the therapeutic compound comprises a synthetic analog of gambogic acid comprising a C6-phenol, a C18-phenol, or combinations thereof. In some embodiments, the therapeutic composition comprises a pharmacophoric motif comprising a caged structure. In some embodiments, the caged structure is a Garcinia xanthone (CGX) motif. In some embodiments, the therapeutic composition has C8-βhydroxyketone functionality. In some embodiments, the therapeutic composition is useful for treatment a solid tumor, a non-solid tumor, a metastasized cancer, or combinations thereof.

In some embodiments, the exposing step occurs for a period of time that is pharmacologically relevant. In some embodiments, the period of time is less than 1 hour, about 1 hour to about 2 hours, about 1 hour to about 3 hours, about 1 hour to about 4 hours, about 1 hour to about 5 hours, about 1 hour to about 6 hours, about 1 hour to about 12 hours, about 6 hours to about 12 hours, about 1 hour to about 24 hours, about 6 hours to about 24 hours, about 12 hours to about 24 hours, about 1 hour to about 48 hours, about 6 hours to about 48 hours, about 12 hours to about 48 hours, about 24 hours to about 48 hours, about 36 hours to about 48 hours, about 1 hour to about 72 hours, about 24 hours to about 72 hours, about 36 hours to about 72 hours, about 48 hours to about 72 hours, about 1 hour to about 96 hours, about 24 hours to about 96 hours, about 48 hours to about 96 hours, about 72 hours to about 96 hours, about 1 hour to about 120 hours, about 24 hours to about 120 hours, about 24 hours to about 120 hours, about 48 hours to about 120 hours, about 72 hours to about 120 hours, about 96 hours to about 120 hours, or greater than about 120 hours, and any intervening ranges therein. In some embodiments, the exposing step occurs for a period of time that constitutes a treatment regimen of the therapeutic compound. In some embodiments, the treatment regimen comprises at least 1 days, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or greater than at least 90 days, and any intervening ranges therein.

In some embodiments, the method further comprises evaluating the circularity of the spheroid after the period of time to determine the cytotoxicity of the therapeutic compound. In some embodiments, evaluating the circularity of the spheroid comprises determining dissolution indices of said spheroid. In some embodiments, wherein determining dissolution indices occurs through bright-field image analysis. In some embodiments, determining dissolution indices occurs through an image analysis software program. In some embodiments, the image analysis software program is ImageJ. In some embodiments, the image analysis software program determines the area and perimeter of the spheroid. In further embodiments, the image analysis software assigns a first value signifying a perfect circle, a near-perfect circle, or a round-like spheroid. In exemplary embodiments, the first value is “1”. In further embodiments, the image analysis software assigns a second value signifying a loss of circularity. In exemplary embodiments, the second value is “0”. In yet further embodiments, the image analysis software program assigns a third value to the spheroid between the first value and the second value indicating the circularity of the spheroid. In some embodiments, the software program has a proviso that the second value signifies a straight line and cannot be achieved given innate circularity of individual cells comprising the spheroid. In some embodiments, the method further comprises using said dissolution indices to plot a dose response curve for said therapeutic compound. In some embodiments, the method further comprises using said dissolution indices to calculate an IC50 value for said therapeutic compound.

In some embodiments, the method further comprises administering to a patient said therapeutic composition. In some embodiments, the patient is suffering from a solid tumor, a non-solid tumor, a metastasized cancer, or combinations thereof.

### BRIEF DESCRIPTION OF THE FIGURES

FIGS. 1A, 1B, 1C, 1D, 1E and IF show spheroidsMARY-X of primary tumor EXPLANT and lung metastasis. FIG. 1A shows spheroids are composed of individual cells with large, fit nuclei as evidenced by mitotic events (arrow heads); FIG. 1B and FIG. 1C show spheroids remain viable in tissue culture up to and beyond 25 days with few visible apoptotic events (arrow heads); FIG. 1D shows metastatic lung emboli are composed of individual cells with large, fit (i.e. mitotic, arrow head) nuclei; FIG. 1E shows in vitro compaction off the individual cells results in spheroids with well-circumscribed edges of both the tumor and lung, respectively (A-E, bar 100 pm); FIG. 1F shows spheroid preparations used in drug screens range in size from slightly 40 pm to slightly 100 μM.

FIGS. 2A and 2B show the pathophysiological gradient of spheroidsMARY-X. FIG. 2A shows in vitro, spheroid cellular mass show differential areas of hypoxia and proliferation; FIG. 2B shows the IBC PDX tumor (tumor emboli 1-4) show distinct areas of high proliferation and hypoxia within each tumor embolus. (A & B 63× magnification).

FIG. 3 shows the effects of FDA-approved anticancer drugs screened using spheroidsMARY-X. Methotrexate and cisplatin show no response retaining well-circumscribed spheroid edges to the control. Low to moderate with slightly distorted spheroid edges response is seen in bortexomib and lapatinib, whereas doxorubicin displays a mixed response where distorted and well-circumscribed spheroids coexist as well as a single cell population, a sign of complete response (bar 50 μm).

FIGS. 4A and 4B show results of high-throughput drug screening using spontaneously-forming spheroidsMARY-X. FIG. 4A shows images from a bright-field microscope (panel insets) are analyzed on ImageJ using an image analysis program where ‘1’ signifies a perfect circle (i.e. intact spheroid) and numbers approaching ‘0’ signify loss of circularity (i.e. dissolute spheroid). Note that ‘0’ signifies a straight line and can never be achieved in image analysis of spheroids given the innate circularity of individual cells. FIG. 4B shows deviations from a well-circumscribed spheroid edge (Control, black arrow heads) are ranked from no (‘no’ response; CR142) as well low (‘low’ response; CR135) to moderate (′low-moderate; MAD 44) response where the formerly well-circumscribed spheroid edge is slightly distorted (bar 100 μm).

FIGS. 5A and 5B contain dose response curve rendering IC50 values of drugs using spontaneously-forming spheroidsMARY-X drug screen. FIG. 5A shows gambogic acid (GA) and MAD28 display ‘complete’ response as there is total dissolution of the formerly intact spheroid with well-circumscribed edges to the single cell state. FIG. 5B shows dissolution indices are determined through image analysis and used to construct a dose-response curve and subsequent IC50 of drugs that displayed response as compared to the standard of care of metastatic breast cancer (i.e. Paclitaxel) (bar 100 μm).

FIGS. 6A, 6B, 6C and 6D contain results of a dual fluorescence viability assay of treated spheroidsMARY-X. FIG. 6A shows Paclitaxel is ineffective at producing a ‘complete’ response by inducing apoptosis of the spheroid cells predominantly located on the outer periphery (arrow head). FIG. 6B and FIG. 6C respectively show that gambogic acid (GA) and MAD28 display ‘complete’ response as there is total dissolution of the formerly compact spheroid with well-circumscribed edges to the single cell state. FIG. 6D contains dose-response curve results in comparable pattern of response (i.e. IC50 of drugs).

FIGS. 7A and 7B show reagents and conditions for the synthesis of CR135 and CR142. FIG. 7A shows 1,4-dibromobutane (5 equiv.), K2CO3 (2 equiv.), DMF, 25-80° C., 8-16 h. FIG. 7B shows PPh3 (5 equiv.), CH3CN, 150° C., microwave irradiation, 2 h; 58% for CR135, 83% for CR142.

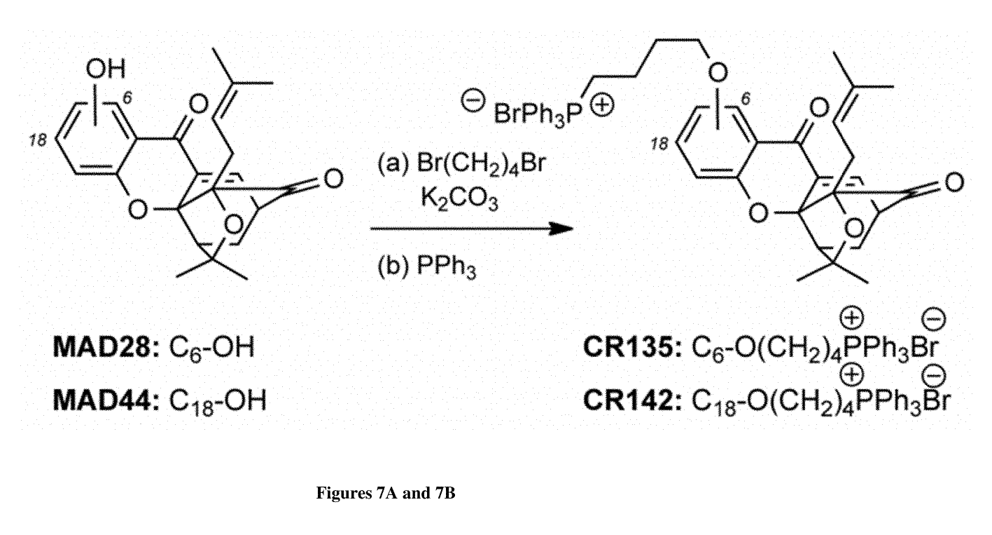

FIG. 8 shows reagents and conditions for the synthesis of CR135.

FIG. 9 shows reagents and conditions for the synthesis of CR142.

FIGS. 10A, 10B, 10C and 10D show proliferative outer spheroidsMARY-X cellular region and dormant tumor cell core. FIGS. 10A, 10B, 10C, and 10D each display spheroidsMARY-X of various sizes, illustrating consistent outer proliferative zone of highly proliferative cells when stained with Ki67 with a quiescent (i.e. dormant) core region of non-proliferative cells that stain positively with hypoxic marker CAIX.

FIG. 11 shows spheroidsMARY-X size distribution. Spheroid size was measured using the Cellometer K2 (Nexcelom Biosciences, Lawrence, Mass.). The Cellometer K2 software provides quantitative measurements of spheroid size distribution.

FIG. 12 shows doxorubicin drug-penetration analysis. SpheroidsMARY-X were exposed to DOX for 3 hours and then orthogonal slices presented (minus volume) following confocal microscopy z-stack analysis.

FIGS. 13A and 13B show PU-H71 treatment of spheroidsMARY-X. FIG. 13A shows vehicle-only treated spheroidsMARY-X. FIG. 13B shows PU-H71 treated spheroidsMARY-X. The vehicle-only treated spheroidsMARY-X are indistinguishable from PU-H71 treated spheroidsMARY-X with regard to well-circumscribed spheroid edges thus indicating no response to treatment with PU-H71.

### DETAILED DESCRIPTION OF THE INVENTION

The invention relates to a high-throughput-compatible method for using an image analysis program to measure circularity of inflammatory breast cancer xenograft-derived “spheroids” (an ex vivo, 3 D cancer tissue mode) as an indicator of response to drug candidates. Circularity or lack thereof is quantitated in a “dissolution index”, which measures drug response. Particularly, as described herein, it has been demonstrated that “spheroids” or the spheroid model described in U.S. Pat. No. 6,998,513, incorporated by reference in its entirety, has a high translational potential and moreover may be used to predict the efficacy and guide the development of new anticancer drugs. Specifically, the invention provides for the first time, a rapid and accurate measurement of compound potency, which, in turn, provides for high-throughput drug screening platforms. Particularly, this invention provides a rapid and reliable method to measure compound cytotoxicity on multicellular spheroids derived from an inflammatory tumor tissue to the compound or a mixture of compounds and optionally using image analysis and dissolution indices to measure the putative antitumor or chemotherapeutic efficacy of the compound or a mixture of compounds. Preferably, the dissolution indices are determined through comparison of simple bright-field image analysis of circularity (intact spheroid) vs. dissolute (single cell populations) and the spheroid dissolution index is determined by applying an image analysis program that measures the circularity of the spheroid.

The limited translational value in clinic of analyses performed on 2-D cell cultures has prompted a shift toward the generation of 3-dimensional (3-D) multicellular systems. Unlike other 3-D screening methods, the present disclosure uses a spontaneously-forming in vitro cancer spheroid model that precisely reflects the pathophysiological features commonly found in tumor tissues and the lymphovascular embolus. Specifically, using the spheroids or spheroids^{MARY-X} system the present disclosure relates to a rapid, inexpensive means to evaluate response following drug treatment where spheroid dissolution indices from bright-field image analyses are used to construct dose-response curves resulting in relevant IC50 values. Using the spheroids or spheroids^{MARY-X} model the Examples herein demonstrate the unique ability of a new class of molecules, containing the caged Garcinia xanthone (CGX) motif, to induce spheroidal dissolution and apoptosis at IC50 values of 0.54+/−0.05 pM for gambogic acid and 1.0+/−0.1 p.M for MAD28. By contrast, treatment of spheroids^{MARY-X} with various currently approved chemotherapeutics of solid and blood-borne cancer types failed to induce any response as indicated by high dissolution indices and subsequent poor IC50 values, such as 7.8+/−3.1 μM for paclitaxel.

Development of in vitro models that more closely resemble living tissue is extremely important in order to decipher cell-cell interactions and signaling especially within the complex tumor tissue environment. Additionally, metastatic disease is the single most crucial reason for morbidity due to cancer and is typically defined by the presence of lymphovascular emboli (LVE), i.e. clumps of cancer cells found within the lymphatics and/or blood vessels. Cancer cells that constitute either the LVE or the tumor are governed by external pressures that vary depending on cell location and microenvironment. Such heterogeneous masses have decreased sensitivity to chemotherapeutic s and, in fact, LVE are viewed as reliable markers for recurrent breast cancer that is resistant to radio- and chemotherapy. 3-D culture models best recapitulate the biological and biochemical heterogeneity of the in vivo embolus/intratumoral cellular mass where, due to external constraints, oxygen, pH and nutritional gradients develop that can significantly impact response to therapeutic agents. Therefore, employing 3-D models in drug development is imperative for successful translation of anticancer therapeutics to the clinic.

As discussed herein, the spontaneously forming spheroid model, spheroidsMARY-X, has an innate ability to form compact tight spheroids where the compaction conveyed by molecular determinants has been proven to contribute to metastatic progression and efficiency. These spheroids mimic both the in vivo metastasis (i.e. LVE) and intratumoral biological/biochemical complexities. In addition, the spheroids^{MARY-X} are not limited in yield and, in conjunction with multi-well plate analyses, may be used to provide a high-throughput (HTP) platform with predictive value for anticancer drug screening and development.

As described herein, spontaneously forming spheroid models, and in particular spheroids^{MARY-X} may be used as a platform for the screening of various small molecule therapeutics. The present disclosure provides the first demonstration of applying dissolution indices to measure drug response of multicellular 3-D spheroids or s spheroids^{MARY-X}. Moreover, using this screening platform, as described herein we demonstrate that synthetic compounds derived from the caged Garcinia xanthones (CGX) family of natural products display potent cytotoxic effects while Federal Drug Administration (FDA)-approved therapies for both solid and blood-borne tumor types fail to elicit a response.

As used herein, “3D cell cultures” or “spheroid cultures” refer to tissues or cells which in vitro exist in 3D microenvironments which may comprise intricate cell-cell and cell-matrix interactions and complex transport dynamics for nutrients and cells. By contrast Standard 2D cell cultures are cell cultures which grow as a monolayer. “3D spheroids” have been reported to more closely resemble in vivo tissue. Therefore, spheroids are sometimes used as models for cell migration, differentiation, survival, and growth. Also, 3D cell cultures reportedly may provide a more accurate depiction of cell polarization, since in 2D, the cells can only be partially polarized. Furthermore, cells grown in 3D may exhibit different gene expression than those grown in 2D.

A real 3D environment is often necessary for cells in vitro to form important physiological structures and functions. The third dimension of cell growth provides more contact space for mechanical inputs and for cell adhesion, which is necessary for integrin ligation, cell contraction and even intracellular signaling. 3D cultures are sometimes used for drug toxicology screening, since the gene expression of the 3 D spheroids may more closely resemble gene expression in vivo. Additionally, some 3D cell cultures have greater stability and longer lifespans than cell cultures in 2D.

There are a large number of commercially available culturing tools that claim to provide the advantages of 3D cell culture. The main categories are extracellular matrices or scaffolds, modified surfaces, rotating bioreactors, microcarriers, magnetic levitation, hanging drop plates, and Magnetic 3D Bioprinting. Scaffold techniques include the use of hydrogels and other materials. Scaffold free techniques employ another approach independent from the use scaffold. For example, they may use low adhesion plate and micropatterned surfaces.

“SpheroidsMARY-X” refers to a specific spontaneously-forming in vitro 3-D cancer spheroid model that precisely reflects the pathophysiological features commonly found in tumor tissues and the lymphovascular embolus. As disclosed in U.S. Pat. No. 6,998,513, hereby incorporated by reference in its entirety, the MARY-X xenograft was established directly from a 45 year old female who presented with a warm and erythematous breast and ill-defined mass which mass was biopsied and diagnosed as inflammatory carcinoma exhibiting florid invasion of dermal lymphatics. Minced portions of the biopsy were washed and implanted subcutaneously in several female SCID and athymic (nude) mice (nu/nu mutants on a BALB/c background). The tumoral xenografts which grew were subsequently transplanted after they reached 1 cm in diameter. Following this procedure, an illustrative stable serial transplantable xenograft designated “MARY-X” was successfully established in both SCID and nude mice. Also, an in vitro culture of a human inflammatory breast cancer xenograft, i.e., spheroids^{MARY-X}, wherein the xenograft grows as a spheroid and which can attach to cell monolayers was previously deposited with the American Type Culture Collection Manassas, Va. on Nov. 29, 2000. As mentioned therein, this xenograft exhibits striking lymphovascular invasion and does not grow as an isolated tumor nodule but grows exclusively within lymphovascular channels. Some of these channels are lymphatics and some are blood vessels. Additionally, the skin overlying the inflammatory xenograft is intensely erythematous just as it is in humans presumably from the lymphovascular obstruction. In both the athymic (nude) mouse and the SCID in vivo models, the inflammatory carcinoma xenograft exhibits a high degree of spontaneous metastasis as early as 6 weeks following local subcutaneous implantation. As further disclosed in U.S. Pat. No. 6,998,513, the unique features of this xenograft allow an in-depth analysis of an inflammatory cancer's vasculature with markers to distinguish lymphatics, angiogenic and old blood vessels. The '513 patent discloses using the xenograft to assess the effects of anti-angiogenic agents on tumorigenicity, intravasation, hypoxia, necrosis, apoptosis, proliferation, and that the vasculature of this inflammatory carcinoma can be examined to gain insights into the angiogenic versus intravasation phenotypes. However, the '513 does not teach or suggest the specific drug screening assays which are the subject of the present disclosure.

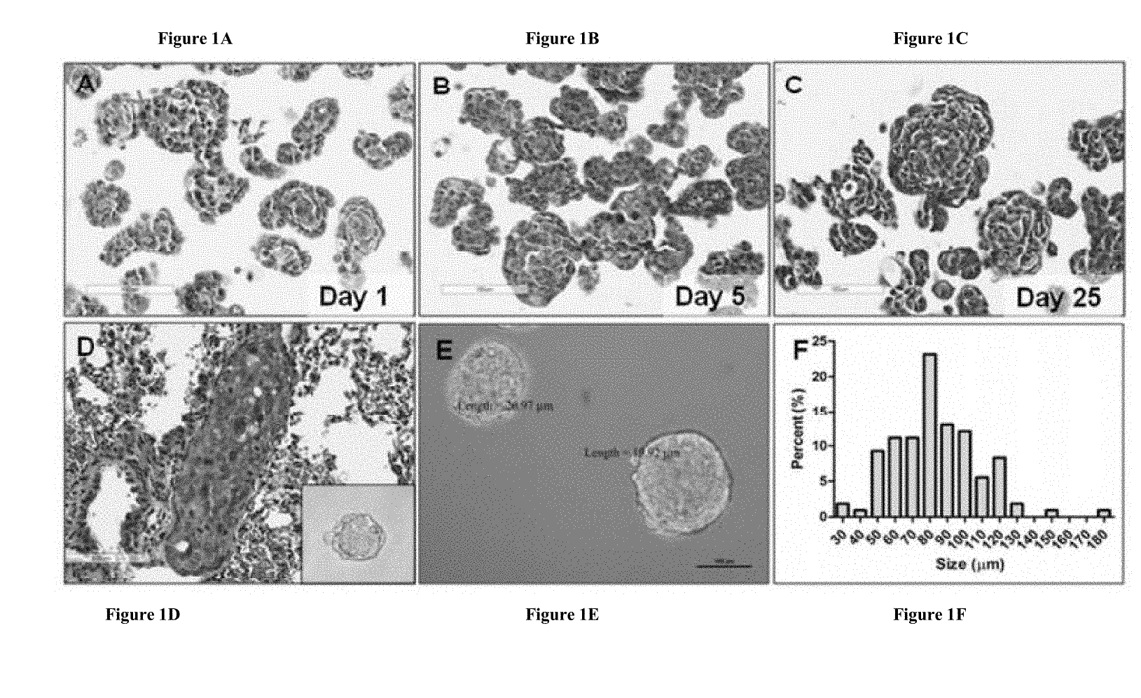

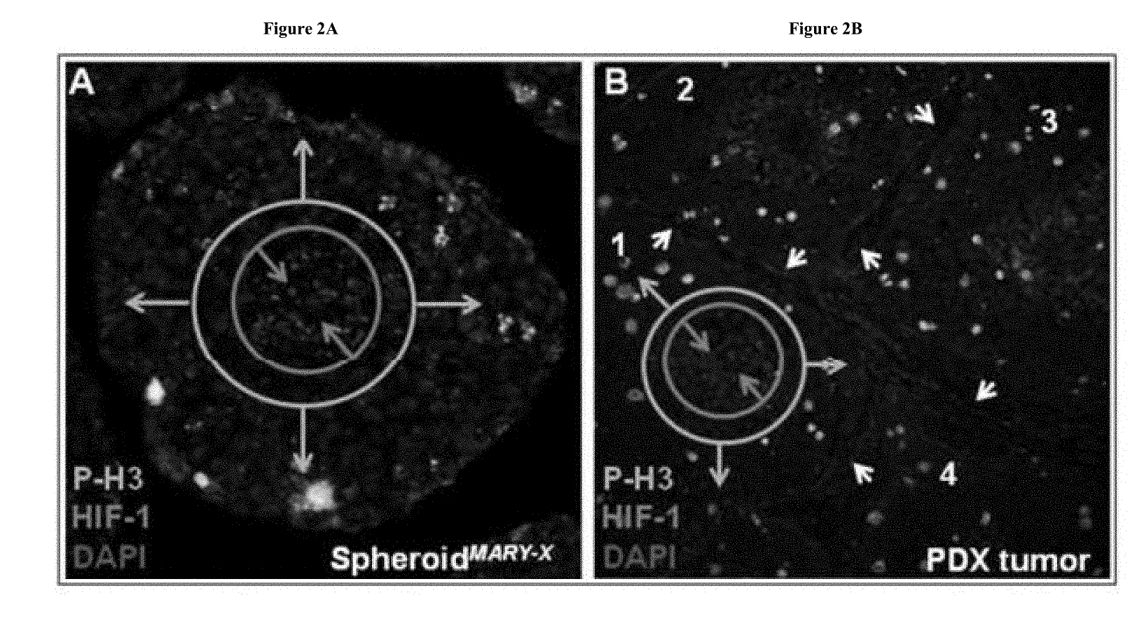

The spontaneously-forming spheroid model, referred to as spheroids or spheroids^{MARY-X}, has distinct advantages over existing art of induced 3-D models. Namely, where 3-D multicellular spheroids are typically developed from formerly traditional 2-D monolayer cell culture, providing only pseudo-representations of multicellular tumor tissue, the spheroids or spheroids^{MARY-X} model has an innate ability to form compact, tight spheroids (see FIG. 1), where the compaction conveyed by innate molecular determinants has been proven to contribute to metastatic progression and efficiency. The spheroids or spheroids^{MARY-X} model mimics both the in vivo metastasis (i.e. LVE) and, intratumoral biological/biochemical complexities (see FIG. 2) a key point for translation of drug response to clinical practice and can be used to develop a patient specific therapeutic regimen. In addition, the spheroids are not limited in yield; a constraint often encountered in existing art 3-D model systems.

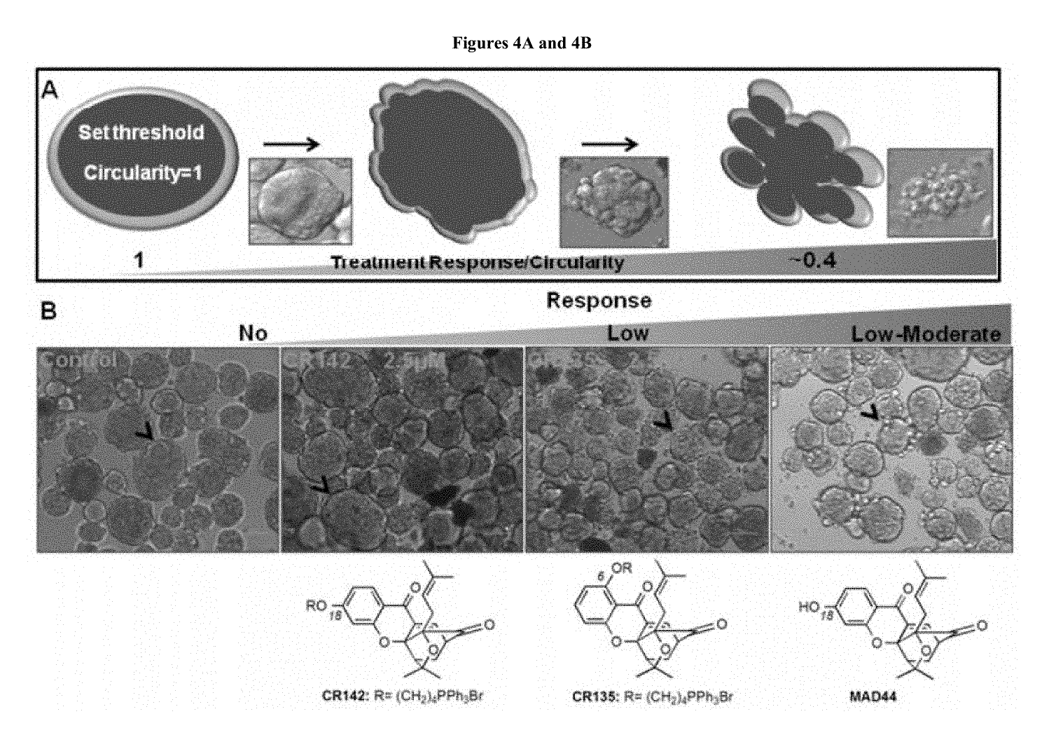

In particular, the invention provides novel methods for determining drug response and calculating drug potency. In drug screens according to the invention spheroids or spheroids^{MARY-X} are seeded in multi-well plates, treated with vehicle only (control) and increasing doses of one or more drugs of interest. Following a 24-hr treatment period each well undergoes analysis to assess dissolution of the spheroids^{MARY-X} (see FIG. 4A, 4B).

In exemplary embodiments the dissolution index is determined by applying an image analysis program on ImageJ that measures the circularity of an object's area and perimeter where ‘1’ signifies a perfect circle (i.e. intact spheroid) and numbers approaching ‘0’ signify loss of circularity (i.e. dissolute spheroid). Note that ‘0’ signifies a straight line and can never be achieved in image analysis of spheroids given the innate circularity of individual cells (see FIG. 4A). The induction of apoptosis correlates with the loss of well-circumscribed edges of the usually tight, compact spheroids, i.e. dissolution is consistent with cell death of the spheroid/embolus.

The Examples infra demonstrate applying a dissolution indices approach to measure drug response of multicellular 3-D spheroids or spheroids^{MARY-X}. Thus this invention overcomes the costly, time-consuming confocal microscopy with multifocal plane analysis and presents a significant advancement over existing art with respect to drug response analyses. Collectively, the spheroids or spheroidsMARY-X system provides a high-throughput (HTP) platform with predictive value for anticancer drug screening and development.

As disclosed herein, the subject invention was used to screen two representative small molecule-based libraries. In order to determine how standard of care drugs would perform in a spheroids screen, the first library consisted of several FDA-approved drugs known to be effective in solid tumor types as well as blood-borne malignancies. Spheroids were added to a multi-well plate (˜30-50 spheroids/well) and then treated with vehicle only (DMSO) and increasing therapeutically-relevant drug doses as follows: bortezomib, lapatinib and doxorubicin (0-2.5 μM), cisplatin (0-10 μM) and methotrexate (0-20 μM).

As used herein, “therapeutically-relevant” is defined as a drug dose that is either recorded in literature or previous experiments as being an effective in vitro dose.

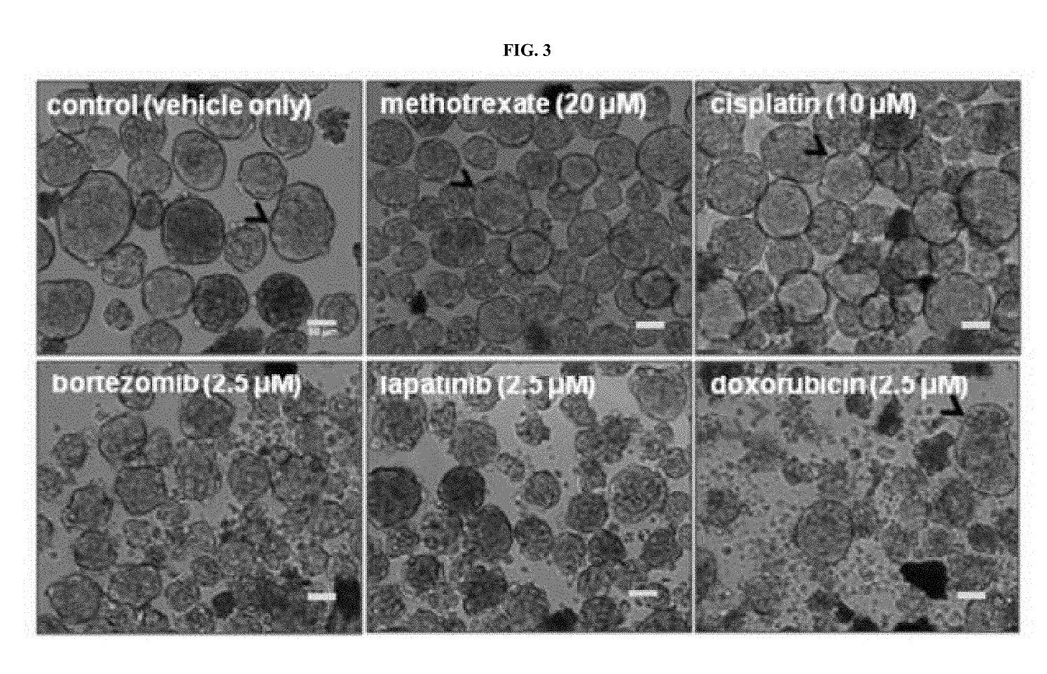

Following a 24-hour treatment period each well underwent analysis to assess dissolution of the spheroids (see FIG. 3). Both methotrexate and cisplatin showed no drug response as the spheroids maintained well-circumscribed edges comparable to the control (see FIG. 3) indicative of spheroid viability. Both bortezomib and lapatinib showed a slight response where spheroid edges become slightly distorted (see FIG. 3). This is in contrast to doxorubicin which showed a mixed response in the treatment of spheroids, where spheroids with well-circumscribed edges (see FIG. 3) coexist with spheroids with significantly distorted edges (see FIG. 3) as well as single cell populations (see FIG. 3), indicative of complete response. Overall, only one of the five FDA-approved drugs, doxorubicin showed potential efficacy in treatment of a relevant model of breast cancer and metastatic disease.

As further disclosed, the second library was composed of two natural products of diverse chemical structure and distinctly different cellular modes of action. Taxol (paclitxael, PTX) is a diterpene that inhibits mitosis by binding to cellular microtubules and is used for the treatment of lung, ovarian and breast cancer. Gambogic acid (GA) is a natural product, derived from traditional ethnomedicine, currently in phase II clinical trials in China as an anticancer agent against non-small cell lung, colon and renal cancers. Compounds MAD28 and MAD44 are synthetic analogues of GA and were synthesized as previously reported in Wang, L. H. et al. Br J Cancer 2014. 110(2): p 341-53, hereby incorporated by reference in its entirety.

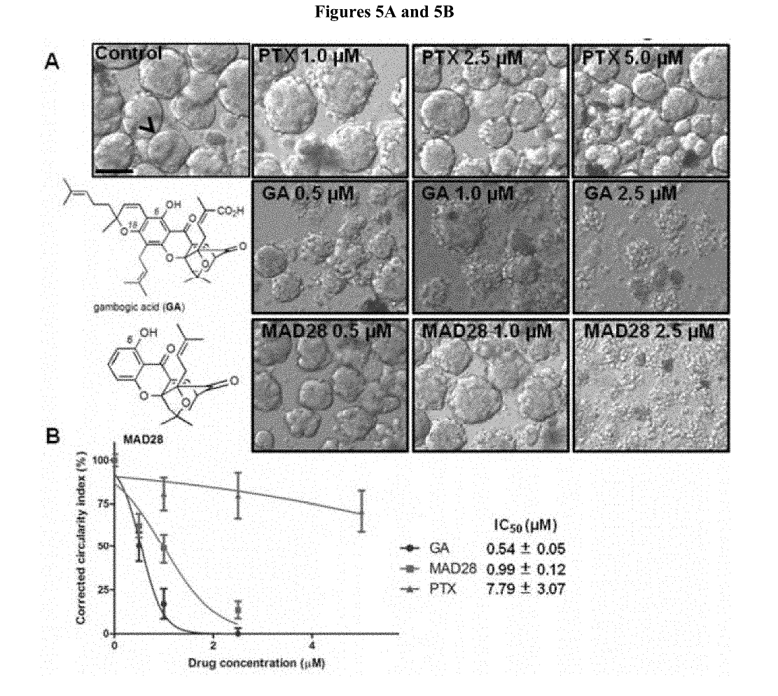

Spheroids were seeded either sparsely (˜30-50/well) for image analysis or more densely (˜100/well) in a replicate plate for further analysis of induction of apoptosis. As the standard treatment of metastatic breast disease and several solid tumor cancer types, Paclitaxel at 1.0, 2.5 and 5.0 μM concentration was included as a control in all drug screens. Synthetic analogues of gambogic acid (GA), CR142, CR135, MAD44 and MAD28, as well as GA were used in this drug screen (see FIG. 4B and FIG. 5A). Each well was treated with vehicle only (DMSO) and increasing doses of compound at 0.5, 1.0 and 2.5 μM concentrations, based on previously reported drug doses of similar structure (see FIG. 4B and FIG. 5A). Only those compounds showing promising response as indicated by a dose-dependent response underwent further image analysis to determine response (i.e. IC50).

Following a 24-hour treatment period each well underwent image analysis to assess dissolution of the spheroids. A well-circumscribed spheroid edge or periphery as seen in the control spheroids was indicative of no drug response, whereas sensitivity is measured by a deviation from a well-circumscribed edge (see FIG. 4A). The response spectrum of analogs tested ranged from ‘no’ to ‘low-moderate’ (see FIG. 4B) with the exception of MAD28 which, comparable to GA, exhibited a ‘complete’ response (i.e. total spheroid dissolution) (see FIG. 5A). Interestingly, paclitaxel, a drug used in clinic for treatment of metastatic breast cancer, was virtually ineffective (see FIG. 5A).

Because the induction of apoptosis is correlated with the loss of the well-circumscribed edges of the usually tight, compact spheroids (i.e. dissolution is consistent with cell death of the spheroid/embolus), the IC50 is determined by applying an image analysis program that measures percent dissolution indices (i.e. circularity of well circumscribed edges of intact spheroid vs. dissolute single cells as shown in FIG. 4A) and using these data to prepare a dose-response curve. Using this method, the calculated IC 50 value for paclitaxel was found to be 7.79+/−3.07 μM. On the other hand, the IC50 values of GA and MAD28 were found to be 0.54+/0.05 μM and 0.99+/−0.12 μM respectively (see FIG. 5B).

Therefore, as described herein, the present disclosure demonstrates that spheroids or spheroids^{MARY-X} have a high translational potential and have predictive value in the development of new anticancer drugs. The methods allow for a rapid and accurate measurement of compound potency, which, in turn, allows the development of high-throughput drug screening platforms.

The terms in this application unless otherwise stated should be accorded their ordinary meaning to one skilled in the relevant art.

### Examples

1. MARY-X Xenograft and In Vitro Spheroids:

Methods

MARY-X is an inflammatory breast cancer (IBC) patient-derived xenograft (PDX). In vivo, the PDX model precisely captures the human IBC signature phenotype of extensive intravastion in situ of the lymphatic and blood vessels by tumor emboli. The IBC spheroids are a cellular derivative of MARY-X tumor explants. These tumor cells form tight compact aggregates of cells termed “MARY-X spheroids” and are presented as spheroids^{MARY-X} herein. These spheroids can be partitioned from the cellular debris by employing cell strainers of varying pore size. A 100 I.A.M cell strainer (Falcon Cell Strainer; Fisher Scientific) was used to exclude spheroids of 100 I.A.M and greater. The filtrate was then passed through a 70 I.A.M and 40 I.A.M cell strainer sequentially, which partitioned the 40-100 I.A.M spheroids that were isolated and subsequently used for all drug screen analyses.

Cells were maintained in minimal essential medium (MEM) containing 10% fetal bovine serum (FBS) and antibiotics (100 U/mL penicillin and 100 μg/mL streptomycin) at 37° C. in an air-5% CO2 atmosphere at constant humidity. All experiments were performed in compliance with the Memorial Sloan Kettering Cancer Center Animal Care and Use Program (Protocol #06-04-006)

Immunofluorescence:

The immunofluorescent detection of P-Histone H3 and Hif1alpha were performed at the Molecular Cytology Core Facility of Memorial Sloan Kettering Cancer Center using Discovery XT processor (Vent a n a Medical Systems).

Phospho-Histone H3: The tissue sections were blocked for 30 minutes in 10% normal goat serum 0.2% BSA in PBS. The primary antibody incubation (rabbit polyclonal P-HH3 (Ser10) antibody (Upstate, cat.#06-570) was used in 1 μg/mL concentrations. The incubation with the primary antibody was done for 4 hours, followed by 32 minutes incubation with biotinylated goat anti-rabbit Ig G (Vector labs, cat #:PK6101) in 7.5 μg/m L dilution. The detection was performed with Blocker D, Streptavidin-HRP D (Ventana Medical Systems), followed by incubation with Tyramide-Alexa Fluor 488 (Invitrogen, cat. #T20922).

Hif1 alpha: The tissue sections were blocked for 30 minutes in 10% normal goat serum 0.2% BSA in PBS. The primary antibody incubation (rabbit Hif1alpha antibody (Chemicon, cat.#AB3883) was used in 10 μg/mL concentrations. The incubation with the primary antibody was done for 5 hours, followed by 60 minutes incubation with biotinylated goat anti-rabbit IgG (Vector labs, cat #:PK6101) in 7.5 μg/mL dilution. The detection was performed with Blocker D, Streptavidin HRP-D (Ventana Medical Systems), followed by incubation with Tyramide Alexa Fluor 594 (Invitrogen, cat. #T20935).

Immunohistochemistry: The immunohistochemical detection of Ki67 and CAIX was performed at the Pathology Core Facility of Memorial Sloan Kettering Cancer Center

Ki-67 (MIB1):

Antigen recovery was conducted using heat retrieval with Citrate buffer pH 6. The primary antibody incubation (Dako Cytomation, Catalog # M7240) was used at a dilution of 1:200. Standard streptavidin-biotin immunoperoxidase method and DAB as chromogen were used.

Carbonic Anhydrase IX (MSKCC):

Antigen recovery was conducted using heat retrieval with Citrate buffer pH 6. The primary antibody incubation (MSKCC) was used at a dilution of 1:500. Standard streptavidin-biotin immunoperoxidase method and DAB as chromogen were used.

Spheroids^{MARY-X} Size Distribution:

Spheroid size was measured using the cellometer K2 (Nexcelom Biosciences, Lawrence Mass.). A 50 uL aliquot of spheroids was loaded into the 3D chamber and assessed on the Cellometer K2. The Cellometer K 2 software provides quantitative measurements of spheroid size distribution.

Spheroids^{MARY-X} Dissolution Indices:

The spheroid dissolution index was determined by applying an image analysis program on ImageJ that measures the circularity of a n object's area and perimeter where ‘1’ signifies a perfect circle (i.e. intact spheroid) and numbers approaching ‘0’ signify loss of circularity (i.e. dissolute spheroid). Note that ‘0’ signifies a straight line and can never be achieved in image analysis of spheroids given the innate circularity of individual cells.

SpheroidsMARY-X Viability Assay: Dual-fluorescent assays were performed on a Cellometer K2 (Nexcelom Biosciences, Lawrence, Mass.) using the viability stains, acridine orange and propium iodide.

Compound Selection: Two representative small molecule-based libraries were used to evaluate the response of spheroidsMARY-X to chemotherapeutic treatment. The first library was composed of five food and drug administration (FDA)-approved drugs that are used for treatment of both solid and blood-borne tumor types: cisplatin, doxorubicin (adriamycin), methotrexate, lapatinib and bortezomid (FIG. 3). Cisplatin is a platinum-containing drug that binds to and cross links DNA. Doxorubicin is an anthrocyclin analogue that intercalates DNA. Methotrexate is a folic acid antagonist that inhibits cell division by interfering with dihydrofolate reductase. Lapatinib is a tyrosine kinase inhibitor that interferes with the epidermal growth factor receptor pathways. Bortezomid is a modified dipeptide that acts as a proteosome inhibitor. Doxorubicin, lapatinib, and methotrexate are all approved for treatment of breast cancer, while doxorubicin is also recommended as adjuvant therapy in instances of nodal involvement post-surgery. The drug, PU-H71, a potent inhibitor of Hsp90 was also tested. This drug was found to be efficacious in preclinical studies for triple-negative breast cancer and is presently in clinical development.

The second library was composed of two natural products of diverse chemical structure and distinctly different cellular modes of action. Taxol (paclitaxel, PTX) is a diterpene that inhibits mitosis by binding to cellular microtubules and is used for the treatment of lung, ovarian and breast cancer. Gambogic acid (GA) is a natural product, derived from traditional ethnomedicine, currently in phase II clinical trials in China as an anticancer agent against non-small cell lung, colon and renal cancers. Compounds MAD28 and MAD44 are synthetic analogues of GA. It has been previously shown that GA, MAD28 and MAD44 localize in mitochondria to induce rapid ROS accumulation and collapse of the mitochondria 1 membrane potential, ultimately leading to release cytochrome c and cell apoptosis. To further drive the delivery of these compounds to mitochondria, MAD28 and MAD44 was functionalized with a triphenylphosphonium salt.

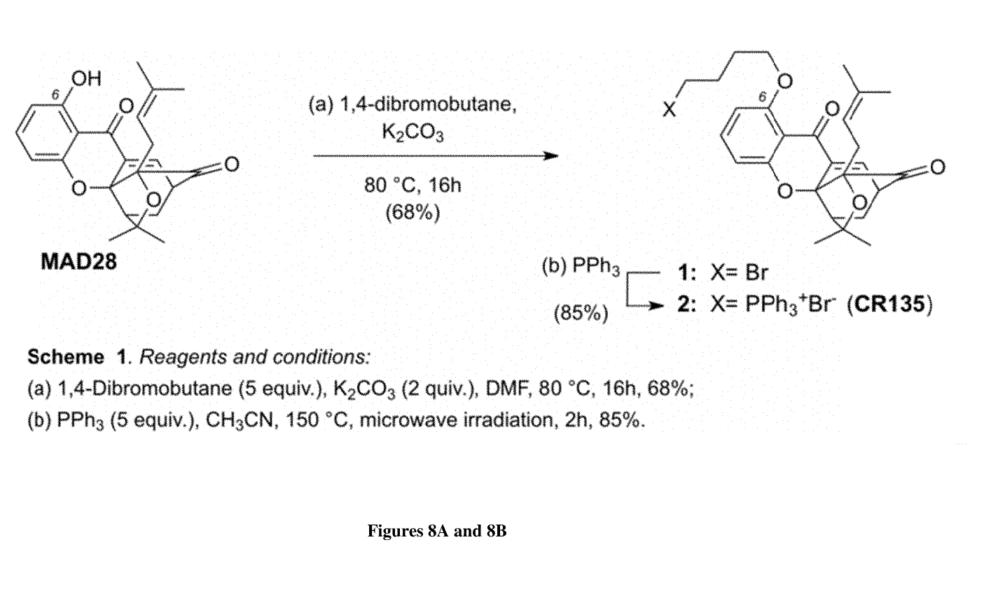

Derivatization of these analogs with a phosphonium salt side chain was performed as described in FIG. 8. Treatment of the A-ring phenolic group with 1,4-dibromobutane (5 equiv.) under basic conditions (K2CO3) produced the corresponding alkyl bromides that, upon reaction with PPh3 (5 equiv.) under microwave conditions produced the desired phosphonium salts CR135 and CR142 in 58% and 83% overall yields.

Drug Screen: The 40-100 μm spheroids were distributed in equal number (˜30-50 spheroids/well) to a multi-well plate. The spheroidsMARY-X was treated with vehicle only (DMSO) and increasing doses of drug of interest in each 24-well plate. Following 24 hr treatment periods each well was imaged and analyzed with ImageJ for measurement of dissolution of the spheroidsMARY-X. The induction of apoptosis is correlated with the loss of the well circumscribed edges of the usually tight, compact spheroids^{MARY-X} i.e. dissolution is consistent with cell death of the spheroid/embolus.

### General Chemical Procedures:

Unless indicated, all commercially available reagents were purchased at the highest commercial quality and were used as received without further purification. All nonaqueous reactions were carried out under argon atmosphere using dry glassware that had been flame-dried under a stream of argon unless otherwise noted. Anhydrous tetrahydrofuran (THF) and dimethylformamide (DMF) were obtained by passing commercially available pre-dried, oxygen-free formulations through activated alumina columns. Flash column chromatography was performed on silica gel (Merck Kieselgel 60, 230-400 mesh). The progress of all the reactions was monitored by thin layer chromatography (TLC) using glass plates precoated with silica gel-60 F254 to a thickness of 0.5 mm (Merck), and compounds were visualized by irradiation with UV light and/or by treatment with a solution of CAM stain followed by heating. 1H NMR and 13C NMR spectra were recorded on a 500 MHz Varian or JEOL instrument. CDCl3 was treated with anhydrous K2 CO3, chemical shifts (δ) are quoted in parts per million (ppm) referenced to the appropriate residual solvent peak reference (CDCl3), with the abbreviations s, d, t, dd, m, denoting singlet, doublet, triplet, doublet of doublets, multiplet, respectively. J=coupling constants given in Hertz (Hz). High-resolution Mass spectra (HRMS) were recorded on a trisector WG AutoSpecQ spectrometer. Gambogic acid was prepared as described in: Guizzunti, G.; Batova, A.; Chantarasriwong, O.; Dakanali, M.; Theodorakis, E. A. “Subcellular localization and activity of gambogic acid” ChemBioChem 2012, 13, 1191-1198, hereby incorporated by reference in its entirety. MAD28 and MAD44 were prepared as reported in: Elbel, K. M.; Guizzunti, G.; Theodorakis, M. A.; Xu, Jing, Batova, A.; Dakanali, M.; Theodorakis, E. A. “A-ring oxygenation modulates the chemistry and bioactivity of caged Garcinia xanthones” Org. Biomol. Chem. 2013, 11, 3341-3348, hereby incorporated by reference in its entirety.

### Synthesis of CR135:

Bromide 1: To a solution of MAD28 (50 mg, 0.13 mmol) in DMF (1 mL), potassium carbonate (36 mg, 0.26 mmol) and 1, 4-dibromobutane (140 mg, 0.65 mmol) were added. The mixture was left stirring at 80° C. during 16 h. Upon completion, the reaction mixture was quenched with water (3 mL) and extracted with diethyl ether (2×10 mL). The combined organic layers were washed with brine, dried over MgSO₄, filtered, and concentrated in vacuum. Purification by flash column chromatography (silica, 30% EtOAc-hexane) gave bromide 1 (45.6 mg, 88.4 μmol, 68% yield). 1: Colorless liquid; Rf=0.5 (50% EtOAc-hexane); 1H NMR (400 MHz, CDCl3) δ 7.38 (t, J=8.4 Hz, 1H), 7.27 (m, 1H), 6.65 (d, J=8.4 Hz, 1H), 6.54 (d, J=8.4 Hz, 1H), 4.48 (t, J=7.2 Hz, 1H), 4.09 (t, J=6.0 Hz, 2H), 3.54 (t, J=6.0 Hz, 2H), 3.46-3.41 (m, 1H), 2.60 (d, J=7.7 Hz, 2H), 2.39 (d, J=9.6 Hz, 1H), 2.32-2.27 (m, 1H), 2.20-2.14 (m, 2H), 2.07-2.00 (m, 2H), 1.69 (s, 3H), 1.66-1.64 (m, 1H), 1.37 (s, 3H), 1.28 (s, 3H), 1.07 (s, 3H); 13C NMR (100 MHz, CDCl3) δ203.61, 175.73, 161.42, 160.60, 136.63, 136.34, 134.95, 132.47, 118.81, 110.58, 110.53, 105.39, 90.06, 84.68, 83.60, 68.16, 48.70, 46.89, 34.09, 30.51, 29.93, 29.51, 29.26, 29.22, 27.76, 25.83, 25.78, 17.21; HRMS calc. for [C27 H32 O5 Br]+(M+H)+515.1428. found 515.1426.

CR135: To a solution of 1 (35 mg, 67.9 μmol) in acetonitrile (1 mL), triphenylphosphine (89 mg, 0.34 mmol) was added. The mixture was stirred under a microwave irradiation for 2 h at 150° C. Upon completion, the reaction mixture was cooled to room temperature and the excess acetonitrile was removed by rotary evaporation. The crude was dissolved in DCM (1 mL) and hexane (10 mL) was added. The solid was filtered and washed with hexane to yield CR135 (44.9 mg, 57.7 μmol, 85% yield). CR135: White solid; Rf=0.1 (20% MeOH-DCM); 1H NMR (400 MHz, CDCl3): 1H NMR (400 MHz, CDCl3) δ 7.88-7.66 (m, 15H), 7.42 (t, J=8.4 Hz, 1H), 7.02 (d, J=6.8 Hz, 1H), 6.63 (d, J=8.4 Hz, 2H), 4.39 (t, J=7.8 Hz, 1H), 4.21 (s, 2H), 4.11-4.00 (m, 2H), 3.42 (dd, J=6.5, 4.4 Hz, 1H), 2.58 (d, J=8.0 Hz, 2H), 2.38 (d, J=9.3 Hz, 1H), 2.31 (dd, J=13.2, 4.4 Hz, 1H), 2.25 (s, 2H), 1.85 (s, 2H), 1.68 (s, 4H), 1.28 (s, 3H), 1.13 (s, 3H), 1.05 (s, 3H); 13C NMR (100 MHz, CDCl3) δ 203.56, 175.83, 161.16, 160.36, 136.86, 135.08, 134.14, 134.04, 131.64, 130.69, 130.57, 119.25, 118.91, 118.40, 110.63, 106.01, 89.98, 84.61, 83.71, 69.05, 48.52, 46.90, 30.48, 29.29, 29.19, 26.02, 25.74, 22.69, 22.19, 20.43, 17.24; HRMS calcd for [C45 H46 O5 P]+(MBr)+697.3077. found 697.3074. Synthesis of CR135 is indicated in FIG. 8.

### Synthesis of CR142

Bromide 3: To a solution of MAD44 (0.1 g, 0.26 mmol) in DMF (2 mL), potassium carbonate (72 mg, 0.52 mmol) and 1, 4-dibromobutane (0.28 g, 1.31 mmol) were added. The mixture was left stirring at room temperature during 8 h. Upon completion, the reaction mixture was quenched with water (10 mL) and extracted with diethyl ether (2×20 mL). The combined organic layers were washed with brine, dried over MgSO4, filtered, and concentrated in vacuo. Purification by flash column chromatography (silica, 30% EtOAc-hexane) gave 3 (0.11 g, 0.22 mmol, 85% yield). 3: Colorless liquid; Rf=0.5 (30% EtOAc-hexane); 1H NMR (400 MHz, CDCl3) δ 7.88 (d, J=8.8 Hz, 1H), 7.38 (d, J=7.0 Hz, 1H), 6.61 (dd, J=8.8, 2.1 Hz, 1H), 6.45 (d, J=2.1 Hz, 1H), 4.44 (t, J=7.4 Hz, 1H), 4.06 (t, J=5.9 Hz, 2H), 3.52-3.45 (m, 3H), 2.60 (d, J=8.3 Hz, 2H), 2.43 (d, J=9.5 Hz, 1H), 2.32 (dd, J=13.5, 4.6 Hz, 1H), 2.10-2.05 (m, 2H), 2.02-1.96 (m, 2H), 1.71 (s, 3H), 1.66 (s, 1H), 1.34 (s, 3H), 1.25 (s, 3H), 1.00 (s, 3H); 13C NMR (100 MHz, CDCl3) δ 203.53, 175.45, 165.83, 161.84, 135.04, 134.94, 133.15, 129.06, 118.81, 113.33, 111.05, 101.49, 90.98, 84.76, 83.65, 77.58, 77.26, 76.94, 67.68, 49.03, 46.91, 33.42, 32.16, 30.65, 29.94, 29.47, 29.30, 27.84, 25.64, 25.47, 22.93, 17.15, 14.37; HRMS (ESI) m/e 515.1428 [M+H]+ calcd for [C27 H32 Br O5]+: 515.1428.

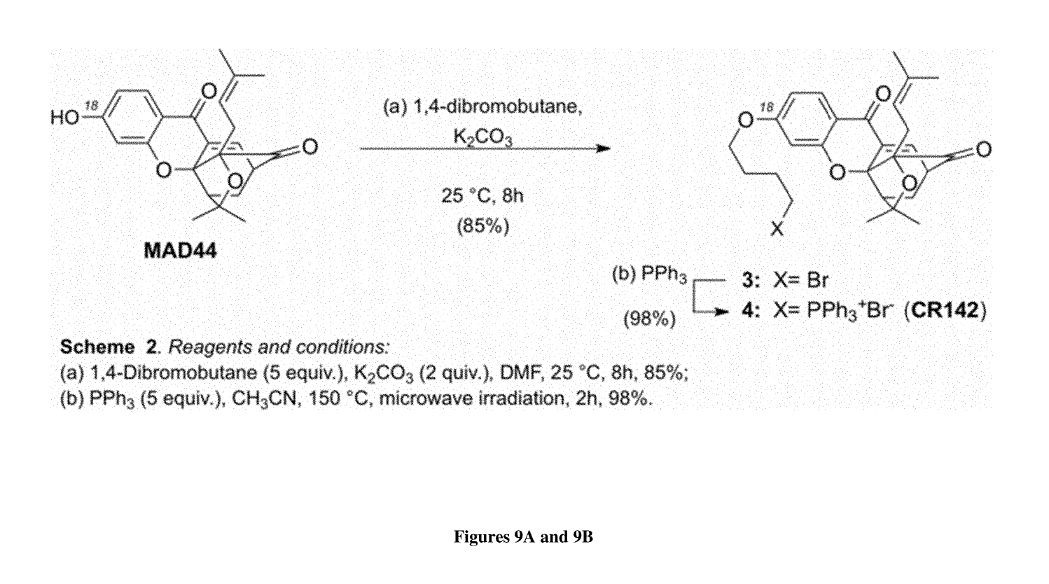

CR142: To a solution of 3 (0.1 g, 0.19 mmol) in acetonitrile (5 mL), triphenylphosphine (0.25 g, 0.97 mmol) was added. The mixture was stirred under a microwave irradiation for 2 h at 150° C. Upon completion, the reaction mixture was cooled to room temperature and the excess acetonitrile was removed by rotary evaporation. The crude was dissolved in DCM (3 mL) and hexane (30 mL) was added. The solid was filtered and washed with hexane to yield CR142 (0.15 g, 0.18 mmol, 98% yield). CR142: White solid; Rf=0.1 (20% MeOH-DCM); 1H NMR (400 MHz, CDCl3) δ 7.89-7.77 (m, 10H), 7.71-7.67 (m, 5H), 7.37 (d, J=7.0 Hz, 1H), 7.17 (d, J=8.8 Hz, 1H), 6.51 (dd, J=8.8, 2.2 Hz, 1H), 6.40 (d, J=2.2 Hz, 1H), 4.43 (t, J=7.2 Hz, 1H), 4.20-4.11 (m, 2H), 4.03 (dd, J=16.8, 12.9 Hz, 2H), 3.46 (dd, J=6.5, 4.5 Hz, 1H), 2.58 (d, J=7.8 Hz, 2H), 2.42 (d, J=9.5 Hz, 1H), 2.35 (s, 1H), 2.33-2.24 (m, 2H), 1.87 (s, 2H), 1.71 (s, 4H), 1.31 (s, 3H), 1.28 (s, 3H), 0.96 (s, 3H); 13C NMR (100 MHz, CDCl3) δ 203.54, 175.46, 165.76, 161.82, 135.27, 135.06, 134.84, 134.01, 133.91, 133.12, 131.13, 130.78, 130.66, 128.97, 118.95, 118.09, 113.31, 111.02, 101.70, 90.96, 84.73, 83.71, 68.36, 67.53, 48.98, 46.92, 38.92, 30.72, 30.56, 29.24, 29.13, 25.64, 25.44, 23.94, 23.21, 19.49, 17.17, 14.30, 11.19; HRMS (ESI) m/e 697.3077 [M-Br]+ calcd for [C45 H46 O5 P]+: 697.3075. Synthesis of CR1242 is indicated in FIG. 9.

### Results

Spontaneous Formation, Morphology and Size Selection of Spheroids^{MARY-X}:

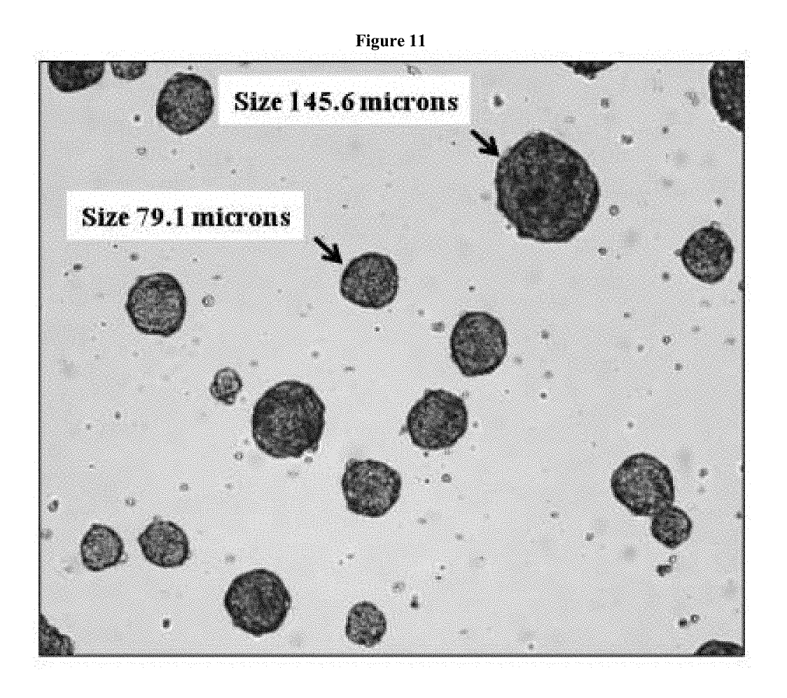

MARY-X, in vitro, is a primary cellular derivative from tumor explants. These tumor cells spontaneously form tight, compact aggregates of cells termed spheroids^{MARY-X} (FIG. 1). Comparable to human inflammatory breast cancer (IBC) emboli, a persistent over-expression of an intact E-cadherin/α, β-catenin axis mediates the compaction of both in vitro and in vivo spheroids^{MARY-X} and tumor emboli, respectively. This persistent over-expression is maintained throughout metastatic progression allowing for spheroids^{MARY-X} derivation from both the primary tumor and lung metastasis (FIG. 1D insert and 1E). For practical purposes, spheroid derivation for drug screening was carried out on spheroids^{MARY-X} obtained from the primary tumor. The spheroids^{MARY-X} ranged in size from as small as 20 uM to as large as 600 uM in diameter. For this Example, spheroids^{MARY-X} ranging in size from ˜40 uM to ˜100 uM were partitioned and used for all drug screening (FIG. 1F and FIG. 11). Drug screens were typically performed within 5 days of the spheroids^{MARY-X} preparation. However, these spheroids^{MARY-X} remain viable in culture as evidenced by fit nuclei displaying mitotic activity in spheroids on day 1 as well as in day 5 and 25 (FIGS. 1A, 1B & 1C) with few apoptotic events seen only in larger spheroids^{MARY-X} at day 25 (FIG. 1C).

Pathophysiological Gradient of Spheroids^{MARY-X}:

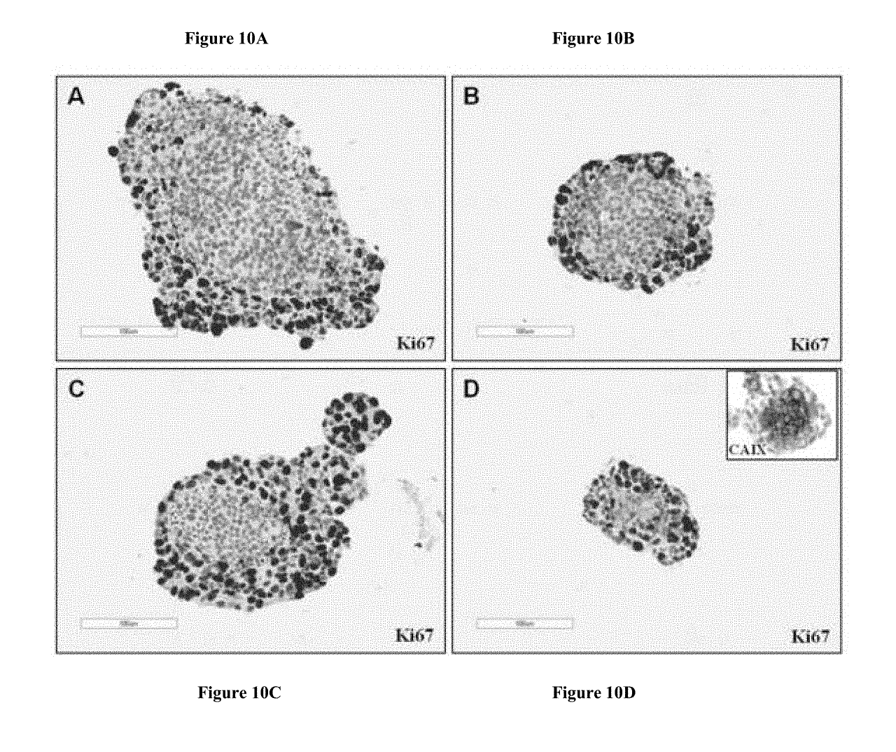

The in vitro spheroids^{MARY-X} (FIG. 2A) is under the physiological constraint diffusion similar to the in vivo solid tumor and/or lymphovascular embolus (FIG. 2B). A region located towards the periphery of the spheroid stains positive with phosphohistone (P-H3), a mitotic marker identifying a proliferative cell supopulation. Cells that are centrally-located stain positive for the hypoxia-inducing factor-1 (HIF-1) with a negative P-H3 status (FIG. 2A) and are, therefore, in a quiescent cellular state. Therapeutics (e.g. chemo/irradiation) that relies on a high proliferation cellular status would be ineffectual on those cells found in the hypoxic region of the cellular mass. Significantly, the spheroids^{MARY-X} recapitulates the biological/biochemical complexity found in vivo (FIG. 2B). In this 2 cm in diameter IBC PDX tumor, all four emboli (FIG. 2B) display a peripheral P-H3 positive cell population with a more centrally-located hypoxic region void o P-H3 positive cells (FIG. 2B; emboli 1 annotated only). These data are consistent with immunohistochemical analyses of spheroids^{MARY-X} with the proliferative marker, Ki67 and hypoxic marker CAIX, (FIG. 10), where highly proliferative cells are found exclusively on the outer perimeter of spheroids^{MARY-X}. The spheroids^{MARY-X} contain pathophysiological gradients consistent with the native in vivo environments of the solid tumor and lymphovascular embolus and therefore provide a very relevant model in the screening of drugs in development.

FDA-Approved Anticancer Therapeutic Drug Screen:

To determine how standard of care drugs would perform in a spheroids^{MARY-X} screen, several FDA-approved drugs known to be effective in solid tumor types as well as blood-borne malignancies, were evaluated. Spheroids were added to a multi-well plate (˜30-50 spheroids/well) and then treated with vehicle only (DMSO) and increasing therapeutically-relevant drug doses as follows: bortezomib, lapatinib and doxorubicin (0-2.5 cisplatin (0-10 μM) and methotrexate (0-20 μM.) Following a 24-hour treatment period each well underwent analysis to assess dissolution of the spheroids^{MARY-X} (FIG. 3). The induction of apoptosis correlates with the loss of well-circumscribed edges of the usually tight, compact spheroids^{MARY-X} i.e. dissolution is consistent with cell death of the spheroid/embolus. Both methotrexate and cisplatin showed no drug response as the spheroids^{MARY-X} maintained well-circumscribed edges comparable to the control (FIG. 3, upper panels), indicative of spheroid viability.

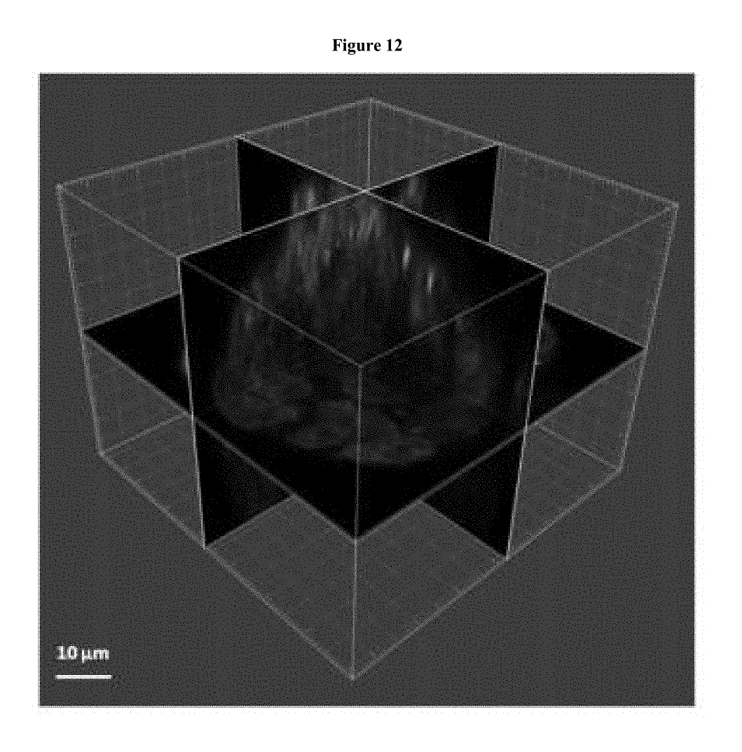

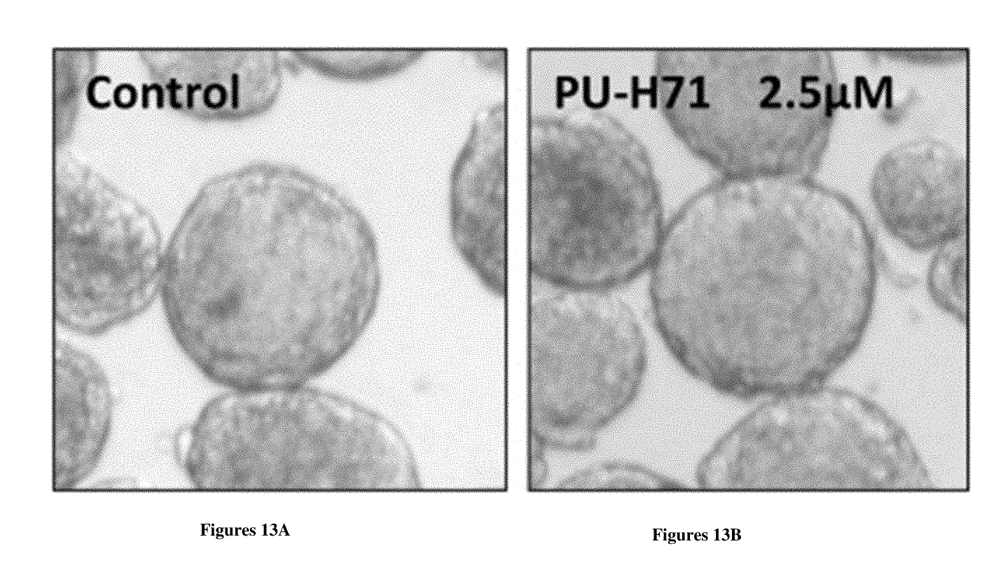

Both bortezomab and lapatinib showed a slight response where spheroid edges become slightly distorted (FIG. 3, lower left and middle panels). This is in contrast to doxorubicin which showed a mixed response in the treatment of spheroids^{MARY-X}, where spheroids with well-circumscribed edges (FIG. 3, lower right panel) coexist with spheroids with significantly distorted edges (FIG. 3, lower right panel) as well as single cell populations (FIG. 3, lower right panel), indicative of complete response. Overall, only one of the five FDA-approved drugs, doxorubicin (TABLE 1, FIG. 12) showed potential efficacy in treatment of a relevant model of breast cancer and metastatic disease. Additionally, tests with PU-H71, a potent Hsp90 inhibitor that is presently in clinical development showed no response when used in the spheroids^{MARY-X} model (FIG. 13), even though in preclinical analysis this drug was found to initiate complete response in several triple-negative breast cancer 2-D culture models.

TABLE 1
Response evaluation of FDA-Approved Drugs

| FDA-Approved Drug | Response |
|---|---|
| Doxorubicin (DOX) | Moderate - Complete |
| Bortezomib | Low - Moderate |
| Lapatinib | Low - Moderate |
| Cisplatin | No |
| Methotrexate | No |

High-throughput drug screen using in vitro spheroids^{MARY-X}. The spontaneously-forming spheroids^{MARY-X} provide an efficient high-throughput platform to screen for efficacy of drugs in development. For this initial investigation focused predominantly on cytotoxicity, spheroids^{MARY-X} were seeded either sparsely (˜30-50/well) for image analysis or more densely (˜100/well) in a replicate plate for further analysis of induction of apoptosis. As the standard treatment of metastatic breast disease and several solid tumor cancer types, paclitaxel at 1.0, 2.5 and 5.0 p.M concentration was included as a control in all drug screens. Synthetic analogues of gambogic acid (GA), CR142, CR135, MAD44 and MAD28, as well as GA were used in this drug screen (FIG. 4B and FIG. 5A). Each well was treated with vehicle only (DMSO) and increasing doses of compound at 0.5, 1.0 and 2.5 μM concentrations, based on previously reported drug doses of similar structure (FIG. 4B and FIG. 5A). Only those compounds showing promising response as indicated by a dose-dependent response underwent further image analysis to determine response (i.e. IC50).

Following a 24-hour treatment period each well underwent image analysis to assess dissolution of the spheroids^{MARY-X}. A well-circumscribed spheroid edge or periphery as seen in the control spheroids^{MARY-X} was indicative of no drug response, whereas sensitivity is measured by a deviation from a well-circumscribed edge (FIG. 4A). The response spectrum of analogs tested ranged from ‘o’ to ‘low-moderate’ (FIG. 4B) with the exception of MAD28 which, comparably to GA, exhibited a ‘complete’ response (i.e. total spheroid dissolution) (FIG. 5A). This response spectrum can provide sufficient qualitative information regarding a small molecule structure activity profile. It is worth noting that GA and MAD28, two CGX analogs containing a C6-phenol group induce a “complete” response. Under identical conditions, MAD44, a structural isomer of MAD28 containing a phenol group at C18 induces a “low-moderate” response. Moreover, functionalization of both MAD28 and MAD44 at the pendant phenol group with a triphenylphosphonium side chain attenuates the compound activity (FIG. 8). Taken together, the results attest to the biological significance of the β-hydroxyketone functionality of MAD28 and GA. Interestingly, paclitaxel, a drug used in clinic for treatment of metastatic breast cancer, was virtually ineffective (FIG. 5A). Because the induction of apoptosis is correlated with the loss of the well-circumscribed edges of the usually tight, compact spheroids^{MARY-X} (i.e. dissolution is consistent with cell death of the spheroid/embolus), the IC50 is determined by applying an image analysis program that measures percent dissolution indices (i.e. circularity of well circumscribed edges of intact spheroid vs. dissolute single cells as shown in FIG. 4A) and using these data to prepare a dose-response curve. Using this method, the calculated IC50 value for paclitaxel was found to be 7.79+/−3.07 μM. On the other hand, the IC50 values of GA and MAD28 were found to be 0.54+/0.05 μM and 0.99+/−0.12 μM respectively (FIG. 5B). Thus, the dissolution indices method allows for the acquisition of quantitative information on the compound activity, further streamlining the use of the spheroids^{MARY-X} assay as a drug screening platform.

Dissolution Indices Vs. Standard Viability Assay:

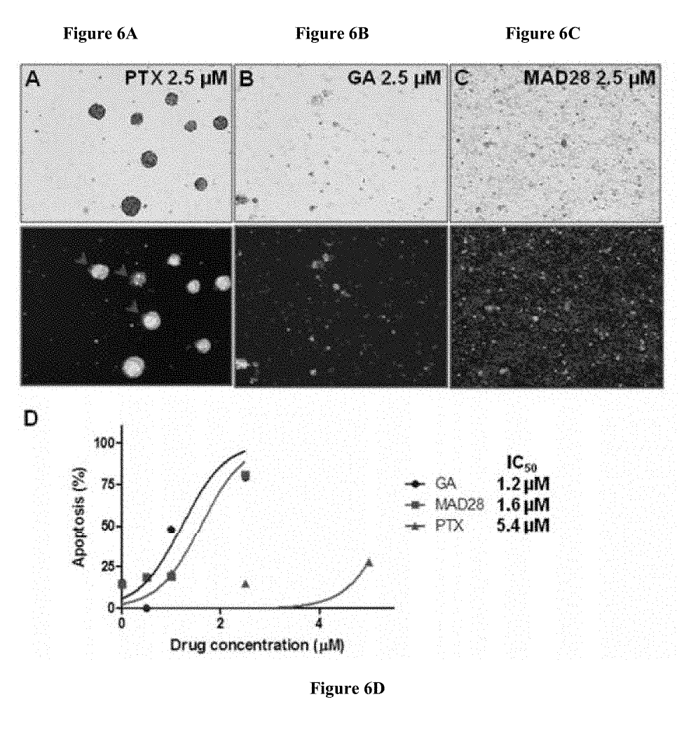

In an effort to validate response data acquired by image analysis (i.e. dissolution indices) an alternate method, traditionally used to determine and quantify cell viability, and was performed on a replicate drug screen. This drug screen reproduced drug dose parameters of paclitaxel (PTX), gambogic acid (GA) and MAD28. Viability was measured by dual fluorescence analysis of propidium iodide (PI) and acridine orange (AO). Following the 24-hour treatment period, spheroids^{MARY-X} were analyzed using the Cellometer K2 in the dual-fluorescence viability assay. The nuclear stains AO and PI were added to the control (vehicle only) and treated spheroids^{MARY-X}, where AO stains both live and dead (green) cells and PI stains only dead (orange) cells with compromised membranes. Fluorescent images presented here are one of eight random fields captured for the viability analyses. Comparable to the previous results, PTX showed little if any activity predominately on the outer periphery of the spheroid (FIG. 6A), whereas both GA and MAD28 displayed complete response as seen by the total dissolution of the formerly compact spheroids^{MARY-X} to a single cell state where the majority of cells are non-viable (dead) (FIGS. 6B and 6C). The IC50 of each drug as calculated from the dose-response curve was 1.2, 1.6 and 5.4 μM for GA, MAD28 and PTX respectively (FIG. 6D), showing a pattern of response comparable to those obtained from the dissolution indices.

The results show that traditional 2-dimensional (2-D) monolayer cell cultures are incapable of recapitulating the heterogeneous and dynamic parameters of the multicellular tumor tissue and lymphovascular embolus, a marker of cancer aggressiveness, recurrence, metastatic progression and most significantly, therapeutic failure. The limitations of traditional 2-D cell cultures to predict in vivo response are particularly important in the drug-screening process and may account for the high failure rate and rising costs of drug discovery. On the other hand, the use of animal models in drug development are time-consuming, expensive and often do not accurately reflect human cancer biology. The gap between 2-D cultures and animal models could be bridged by the development of multidimensional screening methods that, in combination with advances in digital imaging, promise to exponentially increase our understanding on cellular behavior and revolutionize traditional drug discovery. Yet, at present most 3-D multicellular spheroids are typically developed from formerly traditional 2-D monolayer cell culture, providing only pseudo-representations of multicellular tumor tissue. This is in stark contrast to the spheroids^{MARY-X} model, where innate molecular determinants are directly responsible for the spontaneously-forming tight, compact spheroids. The spontaneous formation results in high spheroid yield at a relatively low cost, a distinct advantage over existing 3-D spheroid-generating methods.

The microenvironments dictate pathophysiological parameters of both the tumor tissue and lymphovascular embolus. The spheroid morphology recapitulates these parameters rendering a cellular mass whereby each cell is under unique selective pressure dependent on the location. Each cell or cell population undergoes molecular changes in response to unique environmental (i.e. nutritional and oxygen supply) and selective pressures resulting in pathophysiological gradients where zones of highly proliferative and quiescent/dormant cells (hypoxic region) are established. The spheroids^{MARY-X} contain a peripheral zone where highly proliferative cells reside as well as a population found within the spheroids^{MARY-X} core of quiescent/dormant (i.e. non-proliferative) tumor cells as a result of hypoxic conditions. Although therapeutic resistance can be the result of several molecular factors, it is understood that dormant tumor cells are highly refractory to chemotherapeutics and can result in relapse and late-developing metastases. Developing improved drugs or drug delivery systems to circumvent tumor cell dormancy, as well as in vitro cell models with predictive value is critical in the success of treatment of metastatic disease and cancer in general. The spontaneously-forming in vitro spheroids^{MARY-X} model has captured the biological/biochemical complexities of both the tumor tissue and lymphovascular embolus. The image analysis rendering dissolution indices used to generate dose response curves provide an initial, no cost, rapid and efficient means to screen multiple drug analogs or anticancer therapeutics. The model is also amenable to colorimetric and fluorescent cell viability analyses highlighting the versatility of this high-throughput model system. These key features of the spontaneously-forming spheroids^{MARY-X} provide a relevant model with rapid, efficient and clinically translatable data in drug design and development.

Screening of the library of FDA-approved drugs in the spheroids^{MARY-X} highlights the predictive value of this model in contrast to traditional 2-dimensional (2-D) monolayer cultures, commonly used to determine drug potency prior to costly, laborious in vivo animal efficacy studies. These drugs were chosen because each target cancer-specific vulnerabilities such as DNA synthesis (cisplatin, doxorubicin, and methotrexate), epidermal growth factor receptor (EGFR) (lapatinib) along with the nuclear factor (NF)-KB signaling pathway (bortezomib) known to be constitutively active in many cancer types. Significantly, three of the five drugs used in this drug screen, namely methotrexate, doxorubicin, and lapatinib, are specific for use in breast cancer. Additionally doxorubicin and lapatinib have been approved for treatment of metastatic or late stage breast cancer. At the high end of therapeutically relevant doses as reported in 2-D in vitro models, out of the 5 drugs tested, only bortezomib and lapatinib displayed a low response as indicated by a slightly distorted edge of the spheroids following treatment. On the other hand, treatment with doxorubicin elicited a mixed response where both intact as well as partially dissolute (i.e. single cell population) spheroids^{MARY-X} coexisted. One possible interpretation of this mixed response is that doxorubicin is targeting proliferative cells that compose either smaller spheroids or the outer zone of larger spheroids, thereby leaving behind tumor cells that are still in a predominantly dormant state. This is particularly alarming since after chemotherapy, the remaining dormant tumor cells often lead to resistance, relapse and metastatic disease. Overall, these FDA-approved drugs performed rather poorly in a model system that has captured the pathophysiological features of both the tumor tissue and the lymphovascular embolus.

The spheroids^{MARY-X} screening assay was additionally used to evaluate compounds currently in preclinical development. Paclitaxel, a drug used in the treatment of breast cancer, was used as a control. Interestingly, this drug exhibited only a ‘no to low’ response at a 5.0 μM concentration. In contrast, GA treatment led to complete spheroid dissolution at a 2.5 μM concentration. MAD28, a synthetic analog of GA that contains its pharmacophoric motif (i.e. caged structure and the C8-β-hydroxyketone functionality), was found to have similar potency with complete spheroid dissolution at 2.5 μM. On the other hand, MAD44, a GA analog that contains the caged structure but lacks the β-hydroxyketone functionality induced moderate response at similar concentrations. It was found that derivatizing the phenol group of these compounds with an alkylphosphonium side chain led to significant decrease of activity. Specifically, CR142, an alkylat d derivative of MAD44, showed no response, while CR135, an alkylated derivative of MAD28, induced only moderate response. These data indicate that the spheroids^{MARY-X} assay can be used to identify new anticancer leads as well as provide useful information for structure-function relationship studies.

Rapid measurement of response is imperative in any high throughput drug screening model system. As previously determined the induction of apoptosis is correlated with the loss of ell-circumscribed edges of the usually tight, compact spheroids^{MARY-X} where dissolution into a single cell population is consistent with cell death of the spheroid. Dissolution indices are determined through simple bright-field image analysis of circularity (intact spheroid) vs. dissolute (single cell populations) data which were then used to plot dose response curves and subsequently calculate IC50 values. The IC50 values determined in this screen of library of drugs, was found to be 7.8+/−3.1 uM for paclitaxel, 0.54+/−0.05 uM for GA and 1.0+/−0.1 uM for MAD28. These values were validated with the more commonly used dual fluorescence viability assay of acridine orange and propidium iodide. Comparable IC50 values were measured using the dual fluorescent assay. However, the dissolution indices approach, presented in this study, provides a more rapid and low tech means to measure response in the spheroids^{MARY-X} model system.

In conclusion, this Example describes the use of spheroids^{MARY-X} as a reliable platform for anticancer drug screening. Developed from an inflammatory breast cancer patient-derived xenograft, this assay has distinct advantages over other screening platforms since it creates 3-D cultures that (a) are spontaneously forming and thus do not require extraneous protocols to induce spheroidal morphology; (b) retain the parent tumor phenotype and accurately mimic both the in vivo metastasis (i.e. lymphovascular embolus) and the intratumoral biological complexities of the living tissue; and (c) are high yielding at a low cost and thus amenable to high throughput drug screening. Additionally, this Example describes a rapid, quantitative means to measure drug response and calculate IC50 values using the dissolution indices. The obtained IC50 values correlate well to those derived from a dual fluorescence assay. Most significantly, by using this screening platform, a new family of small molecules were identified, based on the caged Garcinia xanthone (CGX) motif, which induces complete spheroid dissolution at submicromolar concentrations. Based on these results, the CGX motif represents a promising anticancer pharmacophore that can be used to treat both IBC, a cancer with no known targeted therapy, as well as all cancers that do not remain organ-confined (i.e. spheroids^{MARY-X} mimic the lymphovascular embolus).

While this invention has been described with an emphasis upon preferred embodiments, it will be obvious to those of ordinary skill in the art that variations in the preferred compositions and methods may be used and that it is intended that the invention may be practiced otherwise than as specifically described herein. Accordingly, this invention includes all modifications encompassed within the spirit and scope of the invention as defined by the claims that follow.

What is claimed is:

1. A method of screening cytotoxicity of a therapeutic compound comprising:
a. exposing a composition comprising a spontaneously-forming multicellular spheroid derived from a tumor with said therapeutic compound for a period of time; and
b. evaluating the circularity of the spheroid after said period of time to determine the cytotoxicity of said therapeutic compound.

2. The method of claim 1, wherein evaluating the circularity of the spheroid comprises determining dissolution indices of said spheroid.

3. The method of claim 2, wherein determining dissolution indices occurs through bright-field image analysis.

4. The method of claim 2, wherein determining dissolution indices occurs through an image analysis software program.

5. The method of claim 1, wherein said spheroids are derived from a tumor xenograft.

6. The method of claim 1, wherein said spheroid comprises an organoid.

7. The method of claim 1, wherein said spheroid comprises a mammosphere.

8. The method of claim 1, wherein said spheroid is derived from a tumor biopsy.

9. The method of claim 8, wherein said tumor is an inflammatory breast cancer tumor.

10. The method of claim 8, wherein said tumor is a breast, colon, melanoma, lung, skin, pancreatic, liver, brain, ovarian, testicular, prostate, stomach, kidney, tracheal, oral, or esophageal tissue.

11. The method of claim 1, wherein said spheroid comprises a spheroid^{MARY-X}.

12. The method of claim 1, wherein said therapeutic compound is a chemotherapeutic compound.

13. The method of claim 1, wherein said therapeutic compound comprises one of a peptide, polypeptide, nucleic acid, or a small molecule.

14. The method of claim 1, wherein the spheroid is seeded in multi-well plates during the exposing step.

15. The method of claim 1, wherein the period of time is between about 24 hours and about 120 hours.

16. The method of claim 1, wherein said spheroid over-expresses at least one of E-cadherein, alpha-catenin, beta-catenin, or an innate molecular determinant that induces spheroidal morphology.

17. The method of claim 1, further comprising:
c. creating a dose-response curve for said therapeutic compound.

18. The method of claim 1, further comprising:
c. creating an IC150 value for said therapeutic compound.

19. The method of claim 1, further comprising:
c. administering said therapeutic compound to a patient in need thereof.
